(19) United States
(12) Patent Application Publication
(10) Pub. No.: US 2011/0129522 A1
(43) Pub. Date: Jun. 2, 2011

(54) TRANSDERMAL SYSTEM FOR EXTENDED DELIVERY OF INCRETINS AND INCRETN MIMETIC PEPTIDES

(75) Inventors: Keren Mevorat-Kaplan, Ness-Ziona (IL); Galit Levin, Nordiya (IL); Hagit Sacks, Modi'in (IL); Meir Stern, Rohovot (IL)

(73) Assignee: Transpharma Medical Ltd., Lod (IL)

(21) Appl. No.: 13/054,908

(22) PCT Filed: Jul. 21, 2009
(86) PCT No.: PCT/IL09/00713

§ 371 Date: Jan. 19, 2011

Related U.S. Application Data
(60) Provisional application No. 61/082,232, filed on Jul. 21, 2008.

(51) Int. Cl. A61K 38/26; A61K 38/22; A61K 9/70; A61P 3/04; A61P 3/10

(52) U.S. Cl. 424/449; 514/9.7; 514/11.7; 514/5.2; 514/6.9

(57) ABSTRACT

A transdermal patch formulation of a viscous liquid that includes an active agent of incretin or incretin mimetic peptide, a stabilizer, a buffer, a water soluble thickening agent, and a pharmaceutically acceptable carrier. Also, a patch adapted for transdermal delivery of the active agent and having a drug reservoir compartment of the transdermal patch formulation and a system for facilitating transdermal delivery of the active agent through the skin of a subject. The system includes the patch and an apparatus capable of generating a plurality of micro-channels in an area on the subject's skin. The patch and apparatus are used in methods of treatment for reducing blood glucose level, lowering plasma glucagon levels, reducing food intake, or reducing gastric motility in a subject in need of the same by extending the release of the incretin or incretin mimetic peptide in the subject.

### FIELD OF THE INVENTION

The present invention relates to a transdermal system for extended delivery of incretin and incretin mimetic peptides, and to methods of use thereof. Particularly, the present invention relates to a transdermal system comprising an apparatus that generates micro-channels in the skin of a subject in conjunction with a transdermal patch that comprises a drug reservoir layer comprising a formulation which comprises an incretin or incretin-mimetic peptide. The system is useful for extended delivery of incretins and incretin mimetic peptides, particularly of exendin-4, for treating diabetes mellitus and obesity.

### BACKGROUND OF THE INVENTION

The delivery of drugs through the skin provides many advantages. Primarily, such delivery is a comfortable, convenient and noninvasive way of administering drugs. The variable rates of absorption and metabolism encountered in oral treatment are avoided, and other inherent inconveniences, e.g., gastrointestinal irritation and degradation of certain drugs via gastrointestinal enzymes, are eliminated. Transdermal drug delivery theoretically enables a high degree of control over blood concentrations of any particular drug. In reality, many problems remain to be resolved in order to achieve these advantages.

Skin is a structurally complex, relatively thick barrier. Molecules moving from the environment into and through intact skin must first penetrate the stratum corneum. They must then penetrate the viable epidermis, the papillary dermis, and the capillary walls into the blood stream or lymph channels. To be so absorbed, molecules must overcome a different resistance to penetration in each type of tissue. Transport across the skin layers is thus a complex phenomenon. However, it is the cells of the stratum corneum, which present the primary barrier to transdermally administered drugs. The stratum corneum is a thin layer of dense, highly keratinized cells approximately 10-30 microns thick over most of the body. It is believed that the high degree of keratinization within these cells and their dense packing create a substantially impermeable barrier to drug penetration. With many drugs, the rate of permeation through the skin is extremely low and is particularly problematic for high molecular weight drugs such as polypeptides and proteins. Consequently, a means for enhancing the permeability of the skin is desired to effect transport of the drug into and through intact skin.

In order to increase the rate at which a drug penetrates through the skin, various approaches have been adapted, each of which involves the use of either a physical penetration enhancer or a chemical penetration enhancer. Physical enhancement of skin permeation includes, for example, electrophoretic techniques such as iontophoresis or electroporation. The use of ultrasound (or “sonophoresis”) as a physical penetration enhancer has also been studied. Chemical enhancers are compounds that are administered along with the drug (or in some cases the skin may be pretreated with a chemical enhancer) in order to increase the permeability of the stratum corneum, and thereby provide for enhanced penetration of the drug through the skin. However, a major disadvantage exists when using such chemical enhancers as skin damage, irritation, and sensitization are often encountered.

U.S. Pat. No. 6,148,232 to Avrahami describes a device for ablating the stratum corneum of a subject. The device includes a plurality of electrodes, which are applied at respective points on skin of a subject. A power source applies electrical energy between two or more of the electrodes to cause ablation of distinct regions of the stratum corneum (SC), primarily beneath the respective electrodes, and to generate micro-channels. Various techniques for limiting ablation to the stratum corneum are described, including spacing of the electrodes and monitoring the electrical resistance of skin between adjacent electrodes. U.S. Pat. Nos. 6,597,946; 6,611,706; 6,708,060; and 6,711,435 to Avrahami disclose additional devices for ablating the stratum corneum and generating micro-channels so as to facilitate transdermal passage of substances through the skin. The devices are aimed at reducing sensation and minimizing damage to skin underlying the stratum corneum during micro-channel generation.

It has been long appreciated that administration of a therapeutic agent in a manner that does not afford controlled release may lead to substantial oscillation of its levels, at times reaching concentrations that could be toxic or produce undesirable side effects, and at other times falling below the levels required for therapeutic efficacy. A primary goal of the use of devices and/or methods for controlled release is to produce greater control over the systemic levels of therapeutic agents.

Various strategies have been developed aiming at achieving controlled release of a therapeutic agent. Release by controlled diffusion is one of these strategies. Different materials have been used to fabricate diffusion-controlled slow release devices. These materials include non-degradable polymers such as polydimethyl siloxane, ethylene-vinyl acetate copolymers, and hydroxyalkyl methacrylates as well as degradable polymers, among them polylactic/glycolic acid copolymers. Microporous membranes fabricated from ethylene-vinyl acetate copolymers have been used for release of proteins, affording a high release capacity.

An additional strategy for controlled release involves chemically controlled sustained release, which requires a chemical cleavable bond to allow cleavage from a substrate to which a therapeutic agent is immobilized, and/or biodegradation of the polymer to which the agent is immobilized. This category also includes controlled non-covalent dissociation, which relates to release resulting from dissociation of an agent, which is temporarily bound to a substrate by non-covalent binding. This method is particularly well suited for controlled release of proteins or peptides, which are macromolecules capable of forming multiple non covalent, ionic, hydrophobic, and/or hydrogen bonds that afford stable but not permanent attachment of proteins to a suitable substrate.

U.S. Pat. No. 6,275,728 provides a thin film drug reservoir for an electrotransport drug delivery device comprising a hydratable, hydrophilic polymer, said film capable of forming a hydrogel when placed in contact with a hydrating liquid.

International PCT Patent Application WO 2004/039248 to some of the inventors of the present application, disclose a system for transdermal delivery of a dried pharmaceutical composition. The system comprises an apparatus that generates micro-channels in an area on the skin of the subject, and a printed patch comprising a dried pharmaceutical composition comprising a peptide, polypeptide or a protein. The transdermal delivery of the dried peptide, polypeptide or protein into the blood circulation after generation of micro-channels according to WO 2004/039248 depends upon the dissolution of these active agents in exudates released from the micro-channels generated. The transdermal delivery peaks after 2 to 6 hours and declines to baseline levels after 8 to 10 hours from patch application.

International PCT Patent Application WO 2005/056075 to some of the inventors of the present application discloses a system for sustained delivery of peptides, polypeptides, or proteins. The system comprises an apparatus that generates hydrophilic micro-channels in an area on the skin of a subject and a patch comprising a drug reservoir layer comprising a polymeric matrix and a pharmaceutical composition comprising a peptide, polypeptide or protein. WO 2005/056075 further discloses that preferably the patch comprises a drug reservoir layer in a dried form which comprises a dried active agent. The transdermal delivery of the active agent according to WO 2005/056075 depends upon the dissolution of the dry active agent in exudates released from the micro-channels generated, thus achieving sustained transdermal delivery characterized by peak plasma levels of the active agent 6 hours after patch application which decline to baseline levels within 14 hours of patch application. The dried active agent disclosed in both WO 2004/039428 and WO 2005/056075 is first dissolved in a small volume of exudates released from the hydrophilic micro-channels generated, and then it diffuses through the hydrophilic micro-channels into the blood circulation, achieving peak plasma levels of the active agent after a lag time from patch application.

### Exendins

Exendins are peptides that are found in the salivary secretions of various lizards.

Exendin-4 is present in the salivary secretions of Heloderma suspectum (Gila monster), and Exendin-3 is present in the salivary secretions of Heloderma horridum (Mexican Beaded Lizard) (Eng. J., et al., J. Biol. Chem., 265:20259-62, 1990; Eng. J., et al., J. Biol. Chem., 267:7402-05, 1992).

The exendins have some sequence similarity to several members of the mammalian glucagon-like peptide family, with the highest homology, 53%, being to GLP-1.

Similarly to GLP-1, the exendins were found to have insulinotropic activities, although their glucose-lowering effect has a significantly longer duration than GLP-1. Synthetic exendin-4 is currently being approved by the FDA for use in type 2 diabetic patients who are using thiazolinedione alone or in combination with metformin that have not achieved adequate glycemic control. Use of exendin and exendin agonists in treating gestational diabetes mellitus is disclosed in U.S. Pat. No. 6,506,724.

Other uses for exendin-3, exendin-4 and agonists thereof have been disclosed in U.S. Pat. Nos. 6,956,026, 6,872,700, 6,858,576, and 7,138,375. U.S. Pat. No. 6,956,026 discloses use of exendin and analogs thereof for reducing food intake and appetite; U.S. Pat. No. 6,872,700 discloses use of exendin and analogs thereof for suppressing glucagons secretion; U.S. Pat. No. 6,858,576 discloses use of exendin and analogs thereof for reducing gastrointestinal motility; and U.S. Pat. No. 7,138,375 discloses use of exendin and analogs thereof for lowering plasma lipid.

Various administration routes have been suggested for exendins, including intravenous, intraperitoneal, subcutaneous, intramuscular, oral, topical, transmucosal or by pulmonary inhalation. However, due to its peptidic nature, exendin administration is currently performed by subcutaneous injection. Sustained release formulations of exendin have also been suggested. For example, U.S. Pat. Nos. 6,858,576 and 7,138,375 teach repository or “depot” slow release preparations so that therapeutically effective amounts of an exendin may be delivered into the bloodstream over many hours or days following transdermal injection or delivery. U.S. Pat. No. 6,924,264 discloses modified exendins having an exendin linked to one or more polymers such as PEG aiming at improving circulation time, improving resistance to proteolysis, and improving bioavailability and stability.

Kim et al. (Diabetes Care, 30: 1487-1493, 2007) disclose the effects of subcutaneous administration once weekly of a long-acting release (LAR) formulation of a synthetic exendin-4 to diabetic patients. Though the LAR formulation offers a potential 24 hour glycemic control and weight reduction, it still involves the inconvenient subcutaneous injections.

International Patent Application Publication Nos. WO 02/047712 and WO 03/026591 disclose uses of PYY and analogs thereof in treating obesity, decreasing calorie intake, decreasing food intake and decreasing appetite. International Patent Application Publication No. WO 03/057235 discloses uses of PYY and analogs thereof in combination with GLP-1 and analogs thereof for decreasing food intake, decreasing appetite, and for treating or preventing obesity.

There remains an unmet need for systems and methods for transdermal delivery of incretins and incretin mimetic peptides which can substitute the inconvenient parenteral administration, particularly subcutaneous injection, of these peptides.

### SUMMARY OF THE INVENTION

The present invention provides a transdermal patch formulation comprising an incretin or incretin mimetic peptide, a stabilizer, a buffer, a water soluble thickening agent, and a pharmaceutically acceptable carrier, wherein the formulation is in the form of a viscous liquid.

The present invention further provides a transdermal patch comprising a drug reservoir compartment comprising a formulation which comprises an incretin or incretin mimetic peptide, a stabilizer, a buffer, a water soluble thickening agent, and a pharmaceutically acceptable carrier, wherein the formulation is in the form of a viscous liquid.

The present invention further provides a system for facilitating transdermal delivery of an incretin or incretin mimetic peptide comprising an apparatus that generates hydrophilic micro-channels in an area of the skin of a subject and a transdermal patch that comprises a drug reservoir compartment comprising a formulation which comprises an incretin or incretin mimetic peptides, a stabilizer, a buffer, a water soluble thickening agent, and a pharmaceutically acceptable carrier, wherein the formulation is in the form of a viscous liquid.

The present invention further provides uses of said system for reducing food intake and/or appetite, for reducing gastric motility and/or gastric emptying, for reducing blood glucose levels in a subject having diabetes mellitus, and for lowering plasma glucagon in a subject in need thereof.

It is now disclosed, for the first time, that a transdermal patch comprising a drug reservoir compartment which comprises a formulation comprising an exendin, a stabilizer, a buffer, a water soluble etherified cellulose derivative as a thickening agent in an amount ranging from about 0.5% to about 3.5% (w/w) of the formulation, and water, enables achieving therapeutically effective plasma levels of the exendin within a short period of time, typically within 30 minutes to 4 hours, when such a patch is affixed to an area of the skin where micro-channels have been generated. It is to be appreciated that the micro-channels generated by the apparatus of the present invention are open for a defined period of time. Therefore, in order to enable the exendin to be released from the drug reservoir layer of the transdermal patch and to diffuse through the micro-channels into the skin during the time period that the micro-channels remain open, the exendin formulation must comprise a water soluble thickening agent in an amount which produces a viscous liquid. It is now disclosed that the amount of the thickening agent does not exceed 3.5% (w/w) of the formulation.

The present invention is based in part on the findings that non cross-linked water soluble thickening agents are preferable over cross-linked thickening agents as the former provide faster diffusion of the exendin. Among the non cross-linked water soluble thickening agents, water soluble etherified cellulose derivatives were found to be preferable, among which hydroxyethyl cellulose (HEC) was found to be highly preferable as it provides higher pH stability of the formulation and higher diffusion rates of the exendin from the transdermal patch into the blood circulation.

It is further disclosed that affixing the transdermal patch of the present invention to the area of skin where micro-channels are present enables achieving therapeutic plasma levels of the exendin for extended periods of time far longer than those achieved by subcutaneous injection of said exendin. Thereafter, plasma levels of the exendin return to baseline levels within a short period of time, typically within 2 to 4 hours.

The present invention is exemplified by exenatide, a synthetic exendin-4. However, any incretin or incretin mimetic peptide such as exendin-3, GLP-1, PYY, pancreatic peptide (PP), amylin, pramlintide, and analogs or fragments thereof is within the scope of the present invention. The present invention discloses that transdermal delivery of exenatide to animals treated with an apparatus that generates micro-channels decreased their food intake. The decrease in food intake in animals treated transdermally with exenatide was significant and extended for a longer period of time than that observed in animals injected subcutaneously with exenatide. Moreover, transdermal delivery of exenatide abolished the side effects such as nausea and/or vomiting which are apparent when exenatide is injected subcutaneously. The present invention further discloses that transdermal delivery of exenatide is preferable over subcutaneous injections of the long acting release (LAR) exendin-4 formulation as transdermal delivery of exenatide does not require days or weeks to attain therapeutic plasma levels of the peptide nor days or weeks to return to baseline levels.

According to one aspect, the present invention provides a transdermal patch formulation comprising an incretin or incretin mimetic peptide, a stabilizer, a buffer, a water soluble thickening agent, and a pharmaceutically acceptable carrier, wherein the formulation is in the form of a viscous liquid.

According to some embodiments, the incretin or incretin mimetic peptide is selected from the group consisting of an exendin, GLP-1, peptide YY (PYY), pancreatic polypeptide (PP), amylin, pramlintide, and analogs, fragments, derivatives or conjugates thereof. According to additional embodiments, the exendin is selected from the group consisting of exendin-4 as set forth in SEQ ID NO:1, exendin-3 as set forth in SEQ ID NO:2, and analogs or fragments thereof, such as the peptides of SEQ ID NO:3-8. According to a certain embodiment, the exendin is exendin-4 as set forth in SEQ ID NO:1. According to additional embodiments, the incretin or incretin mimetic peptide is selected from the group consisting of SEQ ID NOs:9 to 15. According to further embodiments, the incretin or incretin mimetic peptide, such as exenatide, is present in the formulation in an amount of about 1 mg/gr formulation to about 50 mg/gr formulation, preferably in an amount of about 2 mg/gr formulation to about 10 mr/gr formulation, and more preferably in an amount of about 2 mg/gr formulation to about 5 mg/gr formulation.

According to some embodiments, the stabilizer is a simple or complex carbohydrate. According to further embodiments, the simple or complex carbohydrate is selected from the group consisting of trehalose, mannose, glucose, galactose, raffinose, cellobiose, gentiobiose, sucrose, and a combination thereof. According to a certain embodiment, the stabilizer is trehalose. According to additional embodiments, the incretin mimetic peptide is exenatide, and exenatide and trehalose are present in the formulation in a ratio of about 1:1 (w:w) to about 1:20 (w:w), preferably in a ratio of about 1:2 (w:w) to about 1:10 (w:w), and more preferably in a ratio of about 1:5 (w:w).

According to further embodiments, the buffer is selected from the group consisting of an acetate buffer, citrate buffer, glutamate buffer, and phosphate buffer. According to a certain embodiment, the buffer is an acetate buffer. According to still further embodiments, the buffer maintains the pH of the formulation in the range of about 2.0 to about 8.0, preferably in the range of about 3.0 to 6.0, and more preferably in the range of about 4.9 to about 5.5. According to further embodiments, the concentration of the buffer ranges from about 10 mM to about 30 mM. According to a certain embodiment, the buffer is acetate buffer at a concentration of about 20 mM.

According to yet further embodiments, the water soluble thickening agent is a water soluble etherified cellulose derivative. According to still further embodiments, the water-soluble etherified cellulosed derivative is selected from the group consisting of a hydroxyalkyl cellulose, alkyl cellulose, and alkylhydroxyalkyl cellulose, e.g., hydroxyethyl cellulose, hydroxypropyl cellulose, methylcellulose, hydroxypropyl methylcellulose, and the like. According to a certain embodiment, the water-soluble etherified cellulose derivative is hydroxyethyl cellulose. According to some embodiments, the formulation comprises the water soluble etherified cellulose derivative such as hydroxyethyl cellulose in an amount ranging from about 0.5% to about 3.5% (w/w) of the formulation, preferably from about 1% to about 3% (w/w) of the formulation, and more preferably from about 1% to about 2% (w/w) of the formulation. According to a certain embodiment, hydroxyethyl cellulose is present in the formulation in an amount of about 1.5% (w/w) of the formulation.

According to still further embodiments, the viscous liquid has a viscosity of up to about 10000 centipoise (cps), alternatively of up to about 5000 cps, further alternatively of up to about 4000 cps, 3000 cps, 2000 cps, or yet further alternatively of up to about 1000 cps. According to a certain embodiment, the viscous liquid has a viscosity of about 300 cps to about 900 cps.

According to an exemplary embodiment, the formulation comprises exenatide as set forth in SEQ ID NO:1 in an amount of about 2 mg/gr formulation to about 5 mg/gr formulation, acetate buffer in a concentration of about 20 mM at a pH of about 4.9 to about 5.5, trehalose, wherein the ratio of exenatide and trehalose ranges from about 1:5 (w/w) to 1:10 (w/w), hydroxyethyl cellulose in an amount ranging from about 1.5% (w/w) to 2.5% (w/w) of the formulation, and water.

According to additional embodiments, the formulation can further comprise an agent selected from the group consisting of a preservative, an antioxidant, and a protease inhibitor. The amount of the preservative in the formulation can range from about 1.1 mg/gr formulation to about 4.4 mg/gr formulation. According to a certain embodiment, the formulation comprises m-cresol as a preservative in the amount of about 2.2 mg/gr formulation.

According to another aspect, the present invention provides a patch for transdermal delivery of an incretin or incretin mimetic peptide comprising a drug reservoir compartment which comprises a transdermal patch formulation according to the principles of the present invention.

According to some embodiments, the patch further comprises at least one of the following layers: a backing layer, an adhesive, a rate-controlling layer, a non-rate controlling layer, and a release liner.

It should be understood that the patch according to the invention may be of any suitable geometry provided that it is adapted for stable and, optionally microbiologically controlled, aseptic or sterile storage of the peptide prior to its use.

According to another aspect, the present invention provides a system for facilitating transdermal delivery of an incretin or incretin mimetic peptide through the skin of a subject comprising: (i) an apparatus capable of generating a plurality of micro-channels in an area on the skin of a subject; and (ii) a patch for transdermal delivery of incretin or incretin mimetic peptide according to the principles of the present invention.

According to some embodiments, the apparatus comprises:
a. an electrode cartridge comprising a plurality of electrodes;
b. a main unit comprising a control unit, which is adapted to apply electrical energy between two or more electrodes when the electrodes are in vicinity of the skin, enabling ablation of stratum corneum in a region beneath the electrodes, thereby generating a plurality of micro-channels.

According to additional embodiments, the control unit of the apparatus comprises circuitry to control the magnitude, frequency, and/or duration of the electrical energy delivered to the electrodes, so as to control the current flow or spark generation, and thus the width, depth and shape of the formed micro-channels. Preferably, the electrical energy is at radio frequency.

According to further embodiments, the electrode cartridge is adapted to generate micro-channels having uniform shape and dimensions. Preferably, the electrode cartridge is removable. More preferably, the electrode cartridge is discarded after one use, and as such it is designed for easy attachment to the main unit and subsequent detachment from the main unit.

According to some embodiments, the micro-channels are generated at a density ranging from about 75 micro-channels/cm² to about 450 micro-channels/cm². Preferably the micro-channels are generated at a density ranging from about 150 micro-channels/cm² to about 300 micro-channels/cm².

According to yet further embodiments, the incretin or incretin mimetic peptide to be delivered by the system of the present invention is selected from the group consisting of an exendin, GLP-1, peptide YY (PYY), pancreatic peptide (PP), and analogs, fragments, derivatives and conjugates thereof. According to additional embodiments, the exendin is selected from the group consisting of exendin-4 as set forth in SEQ ID NO:1, exendin-3 as set forth in SEQ ID NO:2, and analogs or fragments thereof. According to a certain embodiment, the exendin is exendin-4 as set forth in SEQ ID NO:1.

According to another aspect, the present invention provides a method for reducing blood glucose level in a subject having diabetes mellitus comprising:
(a) generating a plurality of micro-channels in an area of the skin of said subject; and
(b) affixing a patch to the area of the skin of the subject in which the plurality of micro-channels is present, the patch comprises a drug reservoir compartment comprising a transdermal patch formulation according to the principles of the present invention, thereby reducing blood glucose level in said subject.

According to a further aspect, the present invention provides a method for lowering plasma glucagon in a subject in need of such treatment comprising:
(a) generating a plurality of micro-channels in an area of the skin of said subject; and
(b) affixing a patch to the area of the skin of the subject in which the plurality of micro-channels is present, the patch comprises a drug reservoir compartment comprising a transdermal patch formulation according to the principles of the present invention, thereby lowering plasma glucagon in said subject.

According to still further aspect, the present invention provides a method for reducing food intake in a subject in need of such treatment comprising:
(a) generating a plurality of micro-channels in an area of the skin of said subject; and
(b) affixing a patch to the area of the skin of the subject in which the plurality of micro-channels is present, the patch comprises a drug reservoir compartment comprising a transdermal patch formulation according to the principles of the present invention, thereby reducing food intake in said subject.

According to still further aspect, the present invention provides a method for reducing gastric motility in a subject in need of such treatment comprising:
(a) generating a plurality of micro-channels in an area of the skin of said subject; and
(b) affixing a patch to the area of the skin of the subject in which the plurality of micro-channels is present, the patch comprises a drug reservoir compartment comprising a transdermal patch formulation according to the principles of the present invention, thereby reducing gastric motility in said subject.

According to yet further aspect, the present invention provides a method for extended transdermal delivery of an incretin or incretin mimetic peptide comprising:
(a) generating a plurality of micro-channels in an area of the skin of a subject;
(b) affixing a patch to the area of the skin of the subject in which the plurality of micro-channels is present, the patch comprises a drug reservoir compartment comprising a transdermal patch formulation according to the principles of the present invention; and
(c) achieving a therapeutic plasma concentration of the incretin or incretin mimetic peptide for an extended period of time.

According to some embodiments, generating micro-channels in the skin of a subject is performed by the apparatus described hereinabove.

According to additional embodiments, the incretin or incretin mimetic peptide is selected from the group consisting of an exendin, GLP-1, PYY, PP, amylin, pramlintide, and analogs, fragments, or conjugates thereof. According to a certain embodiment, the exendin is exendin-4 as set forth in SEQ ID NO:1.

According to other embodiments, the water soluble thickening agent is a water soluble etherified cellulose derivative selected from the group consisting of a hydroxyalkyl cellulose, alkyl cellulose, and alkylhydroxyalkyl cellulose, e.g., hydroxyethyl cellulose, hydroxypropyl cellulose, methylcellulose, hydroxypropyl methylcellulose, and the like. According to a certain embodiment, the water soluble etherified cellulose is hydroxyethyl cellulose.

According to some embodiments, the formulation of incretin or incretin mimetic to be used in the methods of the present invention comprises exenatide, trehalose as a stabilizer, wherein the ratio of exenatide and trehalose ranges from about 1:5 (w/w) to about 1:10 (w/w), acetate buffer to maintain the pH of the formulation in the range of about 4.9 to 5.5, hydroxyethyl cellulose in an amount ranging from about 1.5% (w/w) to 2.5% of the formulation, and water.

According to further embodiments, the formulation to be used in the methods of the present invention further comprises an agent selected from the group consisting of a preservative, a protease inhibitor, and an anti-oxidant according to the principles of the present invention.

According to still further embodiments, patch application achieves therapeutic plasma concentrations of the incretin or incretin mimetic peptide for at least about 20% longer period of time than that achieved by subcutaneous injection of said incretin or incretin mimetic peptide. Preferably, patch application achieves therapeutic plasma concentrations of the incretin or incretin mimetic peptide for at least about 50%, 100%, or at least about 200% longer period of time than that achieved by subcutaneous injection of the incretin or incretin mimetic peptide, after which plasma concentration of the incretin or incretin mimetic peptide return to baseline levels, i.e., the levels determined before patch application. It should be appreciated that the patch is typically affixed to the area of the skin of a subject for a predetermined period of time, preferably for at least 6 hours, 8 hours, 10 hours, 12 hours, 14 hours, 16 hours, 18 hours, or more so as to obtain extended delivery of the incretin or incretin mimetic peptide. According to a certain embodiment, the patch is affixed to the area of the skin of a subject for 16 hours. Thus, while subcutaneous administration achieves therapeutic plasma concentrations of exenatide for about 6 to 10 hours, transdermal delivery of exenatide according to the principles of the present invention achieves therapeutic plasma concentrations for at least about 7 hours to at least about 20 hours.

The method for extended transdermal delivery of an incretin or incretin mimetic peptide of the present invention is useful for reducing appetite and/or for lowering plasma lipid in a subject in need of such treatments.

It is to be appreciated that the methods of the present invention achieve therapeutic plasma concentrations of the incretin or incretin mimetic peptide within a short period of time, typically within 30 minutes to 4 hours.

According to another aspect, the present invention provides use of a system which comprises: (i) an apparatus capable of generating a plurality of micro-channels in an area on the skin of a subject; and (ii) a patch comprising a drug reservoir compartment comprising a transdermal patch formulation according to the principles of the present invention, for transdermally delivering the incretin or incretin mimetic peptide to a subject in need of such treatment. The system of the present invention is thus useful for reducing blood glucose level in a subject having diabetes mellitus, for lowering plasma glucagon, for reducing food intake, for reducing gastric motility, and/or for lowering plasma lipid in a subject in need of such treatment.

The present invention will be more fully understood from the following figures and detailed description of the preferred embodiments thereof.

### BRIEF DESCRIPTION OF THE FIGURES

FIG. 1 shows in vitro diffusion of exendin-4 from a formulation comprising exenatide and a water soluble thickening agent: hydroxyethyl cellulose (HEC), carboxymethyl cellulose (CMC), or alginate. The diffusion was determined in a static diffusion cells model.

FIG. 2 shows in vitro diffusion of exendin-4 from a formulation comprising exenatide and a water soluble etherified cellulose derivative: hydroxyethyl cellulose (HEC) or hydroxypropyl cellulose (HPC). The diffusion was determined in a static diffusion cells model.

FIG. 3 shows exendin-4 plasma levels in pigs treated with ViaDerm™ to generate micro-channels and then patches comprising an exenatide formulation with hydroxyethyl cellulose (HEC) or with hydroxypropyl cellulose (HPC) were applied to the treated skin.

FIG. 4 shows exendin-4 serum levels in rats treated with ViaDerm™ and then patches comprising 60 μg or 200 μg of exendin-4 formulated with 1% or 2.5% hydroxyethyl cellulose (HEC) were applied to the treated skin. Control rats were injected subcutaneously with 1 μg exenatide.

FIG. 5 shows exendin-4 plasma levels in pigs treated with ViaDerm™ and then patches comprising 1 mg of exendin-4 formulated in a solution or formulated as a gel with 1% hydroxyethyl cellulose (HEC) were applied to the treated skin. Control pigs were injected subcutaneously with 10 μg exendin-4.

FIG. 6 shows exendin-4 plasma levels in pigs treated with ViaDerm™ to generate micro-channels at a density of 150 micro-channels (MCs)/cm² or 300 MCs/cm², and then patches comprising 0.5 mg or 1 mg of exendin-4 formulated with 1% hydroxyethyl cellulose were applied to the treated skin. Control pigs were injected subcutaneously with 5 μg exenatide or treated with osmotic mini pumps of exendin-4 (5 μg/hour for 10 hours).

FIG. 7 shows exendin-4 plasma levels in pigs treated with ViaDerm™ to generate micro-channels at a density of 150 micro-channels/cm², and then patches comprising 0.5 mg or 1 mg of exenatide formulated with 1.5% hydroxyethyl cellulose were applied to the treated skin. Control pigs were injected subcutaneously with 10 μg exenatide.

### DETAILED DESCRIPTION OF THE INVENTION

The present invention provides systems and methods for delivering incretin or incretin mimetic peptides through treated skin in which micro-channels have been generated.

It is now disclosed that use of a patch comprising a drug reservoir compartment comprising a formulation which comprises an incretin or incretin mimetic peptide, a stabilizer, a buffer, a water soluble non-cross linked thickening agent, and a pharmaceutically acceptable carrier, wherein the formulation is in the form of a viscous liquid, preferably having a viscosity of up to about 10000 cps, when placed on an area of the skin pretreated by an apparatus that generates micro-channels, enables achieving therapeutic plasma concentrations of the incretin or incretin mimetic peptide after a short period of time from the patch application, typically within 0.5 hour to 4 hours; enables achieving extended delivery of the incretin or incretin mimetic peptide with essentially stable plasma levels; and enables achieving rapid decline to baseline levels. These features were not obtained with patches known in the art such as printed patches disclosed in WO 2004/039428 comprising a dried or lyophilized composition or with patches comprising a dried drug reservoir layer that comprises the active agent and a hydrophilic polymer in a dry form as disclosed in WO 2005/056075. The present invention, therefore, provides highly efficient systems and methods for extended transdermal delivery of incretins or incretin mimetic peptides.

The present invention thus provides a transdermal patch formulation comprising an incretin or incretin mimetic peptide, a stabilizer, a buffer, a water soluble thickening agent, and a pharmaceutically acceptable carrier, wherein the formulation is in the form of a viscous liquid.

The present invention further provides a patch for transdermal delivery of an incretin or incretin mimetic peptide comprising a drug reservoir compartment comprising a formulation which comprises an incretin or incretin mimetic peptide, a stabilizer, a buffer, a water soluble thickening agent, and a pharmaceutically acceptable carrier.

The present invention further provides a system for facilitating transdermal delivery of an incretin or incretin mimetic peptide through skin of a subject comprising: (i) an apparatus capable of generating a plurality of micro-channels in an area on the skin of a subject; and (ii) a patch comprising a drug reservoir compartment comprising a formulation comprising an incretin or incretin mimetic peptide, a stabilizer, a buffer, a water soluble thickening agent, and a pharmaceutically acceptable carrier.

The term “micro-channel” as used in the context of the present specification and claims refers to a hydrophilic pathway generally extending from the surface of the skin through all or a significant part of the stratum corneum and may reach into the epidermis or dermis, through which molecules can diffuse. It should be appreciated that after micro channels have been generated in the stratum corneum, the apparatus is removed from the skin, and the active agent diffuses from a patch subsequently placed on the skin into the systemic circulation.

As the micro-channels are aqueous in nature, the system of the present invention is therefore highly suitable for delivery of hydrophilic peptides through the new skin environment, which is created by the ablation of the stratum corneum.

The incretins or incretin mimetic peptides that can be used in the present invention may be naturally occurring incretins or incretin mimetic peptides or modified naturally occurring incretins or incretin mimetic peptides. The peptides may be chemically synthesized using standard techniques for peptide synthesis such as solid-phase peptide synthesis, or may be prepared using DNA techniques known in the art (see, Sambrook et al., Molecular Cloning: A Laboratory Manual, 2^{nd} Ed., Cold Spring Harbor, 1989). The peptides thus produced may or may not be identical to the naturally occurring peptides. Analogs, fragments and conjugates of the naturally occurring incretins or incretin mimetic peptides are encompassed in the present invention so long as they retain one or more of the biological activities of the naturally occurring incretins or incretin mimetic peptides.

The present invention encompasses exendin-4 of the amino acid sequence: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂ as set forth in SEQ ID NO:1; exendin-3 of the amino acid sequence: His Ser Asp Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂ as set forth in SEQ ID NO:2; as well as analogs, fragments, and conjugates thereof well known in the art so long as these analogs, fragments and conjugates retain one or more of the biological activities of the naturally occurring exendin-4, including, but not limited to, lowering plasma glucose in a subject having diabetes mellitus, slowing gastric emptying, reducing food intake, reducing appetite, and reducing plasma glucagon concentrations. The biological activity exerted by an exendin analog, fragment, or conjugate thereof should be similar or higher than that obtained by the naturally occurring exendin-4. It is to be appreciated that exendin-4 produced synthetically is known as exenatide. Thus, exendin-4 and exenatide have the same amino acid sequence and are used interchangeably throughout the specification and claims.

Examples of exendin analogs include:

Exendin-4 (1-30) of the amino acid sequence: His Gly Glu Gly Thr Phe Thr Ser Asp Leu Ser Lys Gln Met Glu Glu Glu Ala Val Arg Leu Phe Ile Glu Trp Leu Lys Asn Gly Gly set forth in SEQ ID NO:3;

Exendin-4 (1-30) amide of the amino acid sequence: His Gly Glu Gly Thr Phe Thr Ser Asp Leu Ser Lys Gln Met Glu Glu Glu Ala Val Arg Leu Phe Ile Glu Trp Leu Lys Asn Gly Gly-NH₂ set forth in SEQ ID NO: 4;

Exendin-4 (1-28) amide of the amino acid sequence: His Gly Glu Gly Thr Phe Thr Ser Asp Leu Ser Lys Gln Met Glu Glu Glu Ala Val Arg Leu Phe Ile Glu Trp Leu Lys Asn-NH₂ set forth in SEQ ID NO:5;

¹⁴Leu, ²⁵Phe exendin-4 of the amino acid sequence: His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser-NH₂ set forth in SEQ ID NO:6;

¹⁴Leu, ²⁵Phe exendin-4 (1-28) amide of the amino acid sequence: His Gly Glu Gly Thr Phe Thr Ser Asp Leu Ser Lys Gln Leu Glu Glu Glu Ala Val Arg Leu Phe Ile Glu Phe Leu Lys Asn-NH₂ set forth in SEQ ID NO:7;

¹⁴Leu, ²²Ala, ²⁵Phe exendin-4 (1-28) amide of the amino acid sequence: His Gly Glu Gly Thr Phe Thr Ser Asp Leu Ser Lys Gln Leu Glu Glu Glu Ala Val Arg Leu Ala Ile Glu Phe Leu Lys Asn-NH₂ set forth in SEQ ID NO:8.

Additional exendin analogs encompassed in the present invention include the analogs disclosed for example, in U.S. Publication No. 2006/0183677 and references cited therein.

Incretins belong to gastrointestinal hormones that increase insulin release from beta cells of the islets of Langerhans after eating, even before blood glucose levels are elevated. Incretins also slow the rate of absorption of nutrients into the blood stream by reducing gastric emptying and may reduce food intake. Incretins inhibit glucagon release from the alpha cells of the Islets of Langerhans. The two main members of incretins that exert these activities are glucagon-like peptide-1 (GLP-1) and Gastric inhibitory peptide (GIP). Both GLP-1 and GIP are rapidly inactivated by the enzyme dipeptidyl peptidase 4 (DPP-4). The present invention thus encompasses incretins and incretin mimetic peptides.

Glucagon-like peptide 1 (GLP-1) of the amino acid sequence: His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg-NH₂ as set forth in SEQ ID NO:9, GIP as set forth in SEQ ID NO:10, and analogs thereof as known in the art are thus included in the scope of the present invention (see, for example, WO 01/98331 and WO 98/08871, the content of which is incorporated by reference as if fully set forth herein). Exendins, by virtue of their homology to GLP-1, and by virtue of their biological activities, are therefore defined as incretin mimetic peptides.

The present invention further encompasses gastrointestinal peptides which include the peptide YY (PYY) as set forth in SEQ ID NO:11 and fragments or analogs thereof, particularly PYY (3-36) as set forth in SEQ ID NO:12; pancreatic peptide (PP) as set forth in SEQ ID NO:13; amylin as set forth in SEQ ID NO:14; pramlintide as set forth in SEQ ID NO:15; and fragments or analogs thereof as known in the art (see for example WO 02/047712; WO 03/026591; and WO 03/057235, the content of which is incorporated by reference as if fully set forth herein). It should be understood that insulin and insulin growth factors are excluded from the scope of the present invention.

The term “analog” as used herein refers to peptides comprising altered sequences by amino acid substitutions, additions, deletions, or chemical modifications of the naturally occurring incretins or incretin mimetic peptides. By using “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution may be made in an amino acid that does not contribute to the biological activity of the peptide. Also included are peptide analogs in which free amino groups have been derivatised to form amine hydrochlorides, p-toluene sulfonylamino groups, carbobenzoxyamino groups, t-butyloxycarbonylamino groups, chloroacetylamino groups or formylamino groups. According to the invention, the peptides can have a free carboxyl group, or alternatively the free carboxyl group can be derivatized to form, for example, amides, salts, methyl and ethyl esters or other types of esters or hydrazides.

The present invention encompasses fragments of incretins or incretin mimetic peptides as well as conjugates of these peptides so long as these fragments or conjugates preserve at least one of the biological activities of the naturally occurring incretins or incretin mimetic peptides.

The term “fragment” as used herein refers to a portion of a peptide or of a peptide analog which retains at least one of the biological activities of the naturally occurring incretins or incretin mimetic peptides.

Included within the scope of the invention are peptide conjugates comprising the peptides of the present invention, analogs or fragments thereof joined at their amino or carboxy-terminus or at one of the side chains via a peptide bond to an amino acid sequence of a different protein. Additionally or alternatively, the peptides of the present invention, analogs, or fragments thereof can be joined to another moiety such as, for example, a fatty acid, a sugar moiety, or any known moiety that facilitate membrane or cell penetration.

According to the invention, the patch comprises at least one drug reservoir compartment or layer, in which the formulation is stored. The formulation according to the present invention comprises an incretin or incretin mimetic peptide and a water soluble etherified cellulose derivative as a thickening agent.

Thickening agents are typically added to liquid formulations to increase the viscosity of the resulting formulation. A formulation having an increased viscosity is beneficial for topical applications where controlled release and/or avoiding run-off are important. The thickening agent according to the principles of the present invention should raise the viscosity of the formulation up to about 1,000 centipoise (cps), alternatively up to about 2,000 cps, 3,000 cps, 4,000 cps, 5,000 cps, 6,000 cps, 7,000 cps, 8,000 cps, 9,000 cps, or further alternatively up to about 10,000 cps. Preferably, the thickening agent of the present invention raises the viscosity of the formulation up to about 1,000 cps. Viscosity is measured using a rotating spindle viscometer. Thus, the formulation of the present invention is in the form of a viscous liquid. Increasing the concentration of the water soluble thickening agent yields a formulation in the form of a solid, which is excluded from the scope of the present invention.

The thickening agents to be used according to the principles of the present invention are water soluble thickening agents selected from the group consisting of hydrophilic biopolymers and hydrophilic synthetic polymers. Among the hydrophilic biopolymers, water soluble cellulose derivatives such as water soluble etherified cellulose derivatives are preferred. The term “water soluble” etherified cellulose derivative as used herein refers to a cellulose derivative that typically has solubility in water in the range of 1 gr/ml to 1 gr/30 ml at room temperature. Examples of etherified cellulose are well known in the art (listed in USP) and include alkyl celluloses, hydroxyalkyl celluloses and alkylhydroxyalkyl celluloses e.g., methylcellulose, hydroxyethyl cellulose, hydroxypropyl cellulose, hydroxypropyl methylcellulose, and the like.

It is to be appreciated that due to the fact that the water soluble etherified cellulose derivatives are not cross-linked, these derivatives are highly preferable as they provide incretin formulations in the form of a viscous liquid of which the viscosity is sufficient enough to prevent run-off of the formulation, yet enables diffusion of the incretin from the patch through the micro-channels.

The patch may comprise one or more drug reservoir compartments or layers. According to the present invention, the drug in the form of a viscous liquid is contained in a compartment or layer. Thus, the terms “drug reservoir compartment” and “drug reservoir layer” are used interchangeably throughout the specification and claims and refer to that part of the laminated structure of the patch which comprises or holds the drug formulation. Typically, the number of drug reservoir compartments or layers is determined by the desired release characteristics. The concentration of the active agent in the different compartments or layers may be varied and the thickness of the different compartments or layers need not be the same. Additionally, the drug reservoir compartment or layer may comprise or hold one or more active agents so as to achieve a desired therapeutic effect.

Typically, the drug reservoir compartments or layers of the present invention are thin, flexible, and conformable to provide intimate contact with a body skin, and are able to release an incretin or incretin mimetic peptide from the reservoir at rates sufficient to achieve therapeutically effective transdermal fluxes of the peptide. Materials to be used for drug reservoir compartment are polyurethanes, polyolefins such as polyethylene and polypropylene, silicone, ethylene-ethacrylate copolymer, ethylene-vinyl acetate copolymer, ethylene-vinyl methylacetate copolymer, polytetrafluoroethylene (“Teflon”), polycarbonate, polyvinylidene difluoride (PVDF), polycarbonate, polyvinylidene difluoride (PVDF), polysulfones, and the like.

According to the invention, the patch may comprise one or more rate or non-rate controlling layers, which are usually microporous membranes. The rate or non-rate controlling layers comprise biopolymers and/or synthetic polymers. The rate or non-rate controlling layers are devoid of an active agent. Representative materials useful for forming rate or non-rate controlling layers include, but are not limited to, polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, cellulose acetate and cellulose nitrate, polytetrafluoroethylene (“Teflon”), polycarbonate, polyvinylidene difluoride (PVDF), polysulfones, and the like.

The various layers contact each other by any method known in the art. One such method is to place layers adjacent to each other and apply pressure to the outer sides of the layers to force the layers together. Another method is to coat the surface of each of the layers to be contacted with a solvent, such as water, before placing the layers together. In this way, a thin portion of each surface will become soluble and/or swollen thereby producing adhesion upon contact. Another method is to use a known adhesive on one or more of the contacting surfaces. Preferably, the adhesive is one that will not interfere with the delivery of the active agent from the drug reservoir layer.

According to the invention, the patch is used to administer an incretin or incretin mimetic peptide, which is present in one or more drug reservoir compartments or layers. The drug reservoir layer may itself have adhesive properties, or the patch may further comprise an adhesive layer attached to the drug reservoir layer. The patch may further comprise a backing layer.

Typically, a backing layer functions as the primary structural element of a transdermal patch and provides flexibility and, preferably, occlusivity. The material used for the backing layer should be inert and incapable of absorbing an active agent or any component of the formulation contained within the drug reservoir layer. The backing layer preferably comprises a flexible and/or elastomeric material that serves as a protective covering to prevent loss of the active agent via transmission through the upper surface of the patch, and will preferably impart a degree of occlusivity to the patch, such that the area of the body surface covered by the patch becomes hydrated during use. The backing layer also prevents dehydration of the drug reservoir layer. The material used for the backing layer should permit the patch to follow the contours of the skin and be worn comfortably on areas of skin such as at joints or other points of flexure, that are normally subjected to mechanical strain with little or no likelihood of the patch disengaging from the skin due to differences in the flexibility or resiliency of the skin and the patch. Examples of materials useful for the backing layer are polyesters, polyolefins including monolayers or coextruded multilayers, polyethylene, polypropylene, vinyliden chloride/vinyl chloride copolymer, ethylene/vinyl acetate copolymer, polyurethanes, polyether amides, and the like. The occlusive backing layer may be covered by an adhesive layer to allow sticking the patch on to the skin

During storage and prior to use, the patch may include a release liner. Immediately prior to use, this layer is removed so that the patch may be affixed to the skin. The release liner should be made from a drug impermeable material, and is a disposable element, which serves only to protect the patch prior to application.

According to the principles of the invention, the formulation comprises a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic agent is administered. Carriers are more or less inert substances when added to a formulation to confer suitable consistency or form to the formulation.

As used herein a “pharmaceutically acceptable carrier” is an aqueous solution or suspension. Examples of aqueous carriers include water, saline and buffered media, alcoholic/aqueous solutions, or suspensions.

To optimize desirable characteristics of a formulation, various additives are included in the formulation. Thus, to improve the stability of the peptides, a suitable stabilizing agent is added. Suitable stabilizing agents include, but are not limited to, most sugars, preferably trehalose, mannitol, lactose, sucrose, and glucose. To produce a pH that is compatible with a particular peptide being used, a suitable buffer is used. Suitable buffers include most of the commonly known and utilized biological buffers, including acetate, phosphate or citrate buffer. A compatible pH is one that maintains the stability of a peptide, optimizes its therapeutic effect or protects against its degradation. A suitable pH is generally from about 2 to about 8, preferably from about 3 to about 6.5, more preferably from about 4 to about 6, and most preferably a suitable pH is about 4.9 to about 5.5. Additionally, protease inhibitors, anti-oxidants, and preservatives, alone or in combination, may be added as well.

The amount of an incretin or incretin mimetic peptide necessary to provide the desired levels in plasma can be determined by methods described herein below and by methods known in the art. Thus, the amount of an incretin or incretin mimetic peptide in a formulation per patch can be varied in order to achieve a desired therapeutic effect, typically the amount does not exceed 50 mg/gr of the formulation.

### Devices for Enhancing Transdermal Delivery of Metabolism Regulatory Peptides

The system of the present invention comprises an apparatus for enhancing transdermal delivery of an incretin or incretin mimetic peptide. According to the principles of the invention the apparatus is used to generate a new skin environment through which an incretin or incretin mimetic peptide such as an exendin, GLP-1, PYY, PP, pramlintide, amylin or an analog, fragment or conjugate thereof is delivered efficiently.

The term “new skin environment” as used herein, denotes a skin region created by the ablation of the stratum corneum and formation of a plurality of micro-channels, using the apparatus of the present invention.

The present invention incorporates devices and techniques for creating micro-channels by inducing ablation of the stratum corneum by electric current or spark generation, preferably at radio frequency (RF), including the apparatus referred to as ViaDerm™ or MicroDerm, as disclosed in one or more of the following: U.S. Pat. Nos. 6,148,232; 5,983,135; 6,597,946; 6,611,706; 6,708,060; WO 2004/039428; WO 2004/03927; WO 2004/03926; WO 2004/112689; Sintov et al., J. Controlled Release 89: 311-320, 2003); the content of which is incorporated by reference as if fully set forth herein.

U.S. Pat. No. 6,148,232 to Avrahami, incorporated by reference as if fully set forth herein, discloses an apparatus for applying electrodes at respective points on skin of a subject and applying electrical energy between two or more of the electrodes to cause resistive heating and subsequent ablation of the stratum corneum primarily in an area intermediate the respective points. Various techniques for limiting ablation to the stratum corneum are described, including spacing of the electrodes and monitoring the electrical resistance of skin between adjacent electrodes.

According to some embodiments, the apparatus for enhancing transdermal delivery of an incretin or incretin mimetic peptide comprises: an electrode cartridge, optionally removable, comprising a plurality of electrodes, and a main unit. The main unit loaded with the electrode cartridge is also denoted herein ViaDerm.

The control unit is adapted to apply electrical energy to the electrodes typically by generating current flow or one or more sparks when the electrode cartridge is in vicinity of the skin. The electrical energy in each electrode within the electrode array causes ablation of stratum corneum in an area beneath the electrode, thereby generating a plurality of micro-channels. Preferably, the electrical energy is of Radio frequency (RF).

The control unit comprises circuitry which enables to control the magnitude, frequency, and/or duration of the electrical energy delivered to an electrode, in order to control current flow or spark generation, and consequently to control the dimensions and shape of the resulting micro-channel. Typically, the electrode cartridge is discarded after one use, and as such is designed for easy attachment to the main unit and subsequent detachment from the unit.

To minimize the chance of contamination of the cartridge and its associated electrodes, attachment and detachment of the cartridge is performed without the user physically touching the cartridge. Preferably, cartridges are sealed in a sterile cartridge holder, which is opened immediately prior to use, whereupon the main unit is brought in contact with a top surface of the cartridge, so as to engage a mechanism that locks the cartridge to the main unit. A simple means of unlocking and ejecting the cartridge, which does not require the user to touch the cartridge, is also provided.

Optionally the electrode cartridge may further comprise means to mark the region of the skin where micro-channels have been created, such that a patch can be precisely placed over the treated region of the skin. It is noted that micro-channel generation (when practiced in accordance with the techniques described in the above-cited US patents to Avrahami et al. and patent applications, assigned to the assignee of the present patent application) does not generally leave any visible mark, because even the large number of micro-channels typically generated are not associated with appreciable irritation to the new skin environment.

According to some embodiments, current may be applied to the skin in order to ablate the stratum corneum by heating the cells. According to other embodiments, spark generation, cessation of spark generation, or a specific current level may be used as a form of feedback, which indicates that the desired depth has been reached and current application should be terminated. For these applications, the electrodes are preferably shaped and/or supported in a cartridge that is conducive to facilitating ablation of the stratum corneum and the epidermis to the desired depth, but not beyond that depth. Alternatively, the current may be configured so as to ablate the stratum corneum without the generation of sparks.

Preferred embodiments of the present invention typically incorporate methods and apparatus described in U.S. Pat. No. 6,611,706 entitled “Monopolar and bipolar current application for transdermal drug delivery and analyte extraction,” which is assigned to the applicant of the present invention and incorporated by reference as if set forth herein. For example, U.S. Pat. No. 6,611,706 describes maintaining the ablating electrodes either in contact with the skin or up to a distance of about 500 microns therefrom. Thus, the term “in vicinity” of the skin as used throughout the specification and claims encompasses a distance of 0 to about 500 microns from the electrodes to the skin surface. The application further describes spark-induced ablation of the stratum corneum by applying a field having a frequency between about 10 kHz and 4000 kHz, preferably between about 10 kHz and 500 kHz.

According to the present invention, the cartridge supports an array of electrodes, preferably closely-spaced electrodes, which act together to produce a high micro-channel density in an area of the skin under the cartridge. Typically, however, the overall area of micro-channels generated in the stratum corneum is small compared to the total area covered by the electrode array.

According to some embodiments, the diameter of the electrodes is in the range of about 30 to about 150 microns. According to certain exemplary embodiments, the diameter of the electrodes within an electrode array is in the range of about 40 to about 100 microns. According to other embodiments, the length of the electrodes is in the range of about 30 to about 500 microns. According to some embodiments, the length of the electrodes is in the range of about 40 to about 150 microns. According to a certain exemplary embodiment, the length of the electrodes is of about 50 microns.

According to other embodiments of the present invention, a concentric electrode set is formed by employing the skin contact surface of the cartridge as a return path for the current passing from the electrode array to the skin. Preferably, the cartridge has a relatively large contact surface area with the skin, resulting in relatively low current densities in the skin near the cartridge, and thus no significant heating or substantial damage to the skin at the contact surface.

In proximity to each electrode in the electrode array, by contrast, the high-energy applied field typically induces very rapid heating and ablation of the stratum corneum.

### Uses of the Transdermal System

The present invention further provides a method for extended delivery of an incretin or incretin mimetic peptide using a transdermal delivery system according to the principles of the present invention. Typically, the procedure for forming new skin environment comprises the step of placing over the skin the apparatus for generating a plurality of micro-channels. Preferably, prior to generating the micro-channels, the treatment sites will be swabbed with sterile alcohol pads. More preferably, the site should be allowed to dry before treatment.

According to certain exemplary embodiments of the present invention, the type of apparatus used to generate micro-channels is disclosed in for example U.S. Pat. Nos. 6,148,232 and 6,708,060; WO 2004/039428 and continuations thereof, and in Sintov et al., ibid, the content of which is incorporated by reference as if fully set forth herein. The apparatus containing the electrode array is placed over the site of treatment, the array is energized by RF energy, and treatment is initiated. In principle, the ablation and generation of micro-channels is completed within seconds. The apparatus is removed after micro-channels are generated at limited depth, preferably limited to the depth of the stratum corneum and the epidermis. A patch according to the principles of the present invention is attached to the new skin environment.

The present invention provides a method for extended transdermal delivery of an incretin or incretin mimetic peptide, the method comprises the following steps:
(i) generating a plurality of micro-channels in an area of the skin of a subject in a subject in need thereof;
(ii) affixing a patch to the area of the skin in which the plurality of micro-channel is present, the patch comprises a drug reservoir layer comprising a transdermal patch formulation which comprises an incretin or incretin mimetic peptide, a stabilizer, a buffer, a water soluble thickening agent, and a pharmaceutically acceptable carrier; and
(iii) achieving a therapeutically effective plasma concentration of the incretin or incretin mimetic peptide for an extended period of time.

As defined herein “therapeutically effective plasma concentration” means a concentration of an incretin or incretin mimetic peptide, such as an exendin, GLP-1, GIP, PYY, PP, or an analog, fragment or conjugate thereof, which results in a therapeutic effect.

The term “therapeutic” is meant to include amelioration of the clinical condition of a subject and/or the protection, in whole or in part, against a pathological condition or disease.

According to an exemplary embodiment, the exendin is exendin-4 as set forth in SEQ ID NO:1. As disclosed herein below, exendin-4 transdermally administered to rats or pigs by the system of the present invention increased plasma levels of exendin-4 within 30 minutes to two hours after patch application, and such levels were maintained for about 12 to 14 hours, after which the plasma levels of exendin-4 returned to baseline levels (see Examples 2 to 6 herein below). In contrast, subcutaneous administration of exendin-4 resulted in an increase of plasma levels of the peptide 10 minutes to 1 hour after injection, and the plasma levels of exendin-4 returned to baseline levels within 3 to 5 hours. Thus, transdermal delivery according to the present invention provides sustained delivery of exendin-4.

According to some embodiments, a therapeutically effective plasma concentration of about 5 pg/ml to about 500 pg/ml of exendin-4 be achieved, preferably of about at least 25 pg/ml to about 250 pg/ml, alternatively of about 150 pg/ml. According to additional embodiments, the therapeutically effective plasma concentration is maintained for at least 7 hours, alternatively for at least 20 hours.

The present invention thus encompasses patches comprising a formulation comprising one or more peptides selected from the group consisting of an exendin, a member of GLP family including, but not limited to, GLP-1, PYY, PP, amylin, and pramlintide (see, for example, U.S. Pat. Nos. 5,686,411; 5,998,367; 6,410,511; and 6,610,824, the content of which is incorporated by reference as if fully set forth herein), and analogs or fragments thereof, which are stored within the drug reservoir compartment or layer, said formulation further comprises a water soluble etherified cellulose derivative. Use of such patches in conjunction with the apparatus of the present invention results in achieving therapeutic plasma concentrations of incretin or incretin mimetic peptide for extended periods of time, e.g., for more than 7 hours, after which plasma levels return to basal plasma concentrations before patch application.

Additionally, a therapeutic plasma concentration of an incretin or incretin mimetic peptide is determined by the clinical state of a subject. Thus, based on the clinical state of a subject, a clinician would determine a therapeutic concentration of an exendin, GLP-1, PYY, PP, amylin, pramlintide or an analog or fragment thereof as known in the art. Similarly, the duration of treatment or duration of exposure to the incretin or incretin mimetic peptide will be determined by the clinician taking into consideration the disease to be treated, as well as secondary factors including the gender, age, and general physical condition of the patient.

The method of the present invention for extended transdermal delivery of an incretin or incretin mimetic peptide is useful for reducing blood glucose level in patients having diabetes mellitus (including type 1 and type 2 and gestational diabetes mellitus); for reducing gastric motility and/or gastric emptying; for reducing food intake and/or appetite in subjects in need thereof such as obese subjects or diabetic patients; for lowering plasma glucagon levels; and/or for lowering plasma lipid.

It is to be understood that the incretin or incretin mimetic peptide can be provided alone or in combination with another therapeutic agent. Thus, for example, exendin-4 can be administered transdermally by the methods of the present invention in combination with GLP-1 and/or PYY, such as PYY(3-36) or in combination with orally administered metformin and/or sulfonylurea.

Having now generally described the invention, the same will be more readily understood through reference to the following examples, which are provided by way of illustration and are not intended to be limiting of the present invention.

### Example 1

### In-Vitro Diffusion of Exenatide Formulated with Different Thickening Agents

In vitro diffusion of exenatide from formulations containing different thickening agents was measured in static diffusion cells model. Polyethersulfone filter membranes (0.45 μm) were placed in static diffusion cells between the donor and acceptor chambers. The donor chamber was filled with an exenatide formulation to be tested and the acceptor chamber was filled with acetate buffer. Exenatide cumulative release was measured at the indicated time periods using HPLC quantitative method.

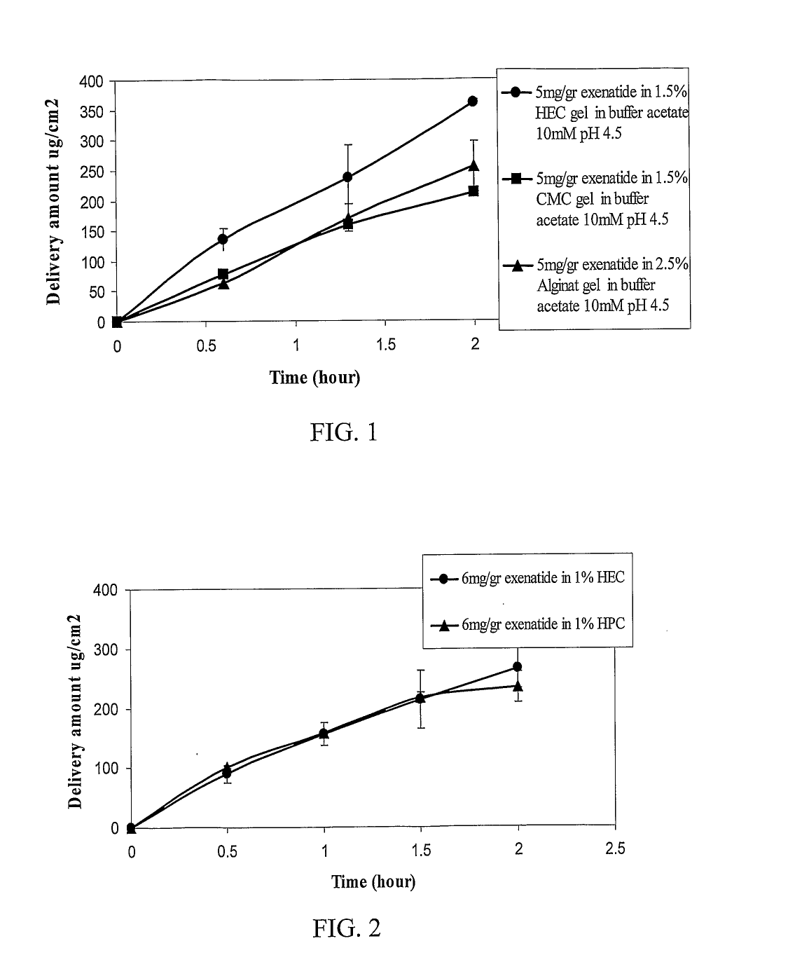

FIG. 1 shows in vitro exenatide diffusion from formulations comprising hydroxyethyl cellulose (HEC), carboxymethyl cellulose (CMC) or alginate. The results show that in vitro diffusion from exenatide formulation containing HEC was found to be higher than from exenatide formulation containing CMC or alginate.

FIG. 2 shows in vitro exenatide diffusion from formulations comprising hydroxyethyl cellulose (HEC) or hydroxypropyl cellulose (HPC). The results show that in vitro diffusion from exenatide formulation containing HEC was found to be similar to that from exenatide formulation containing HPC.

### Example 2

### Transdermal Delivery of Exenatide Formulated with Hydroxyethyl Cellulose or Hydroxypropyl Cellulose—in Pigs

To evaluate whether extended release of exenatide can be achieved by transdermal delivery through micro-channels, pigs were treated with the ViaDerm™ apparatus to generate micro-channels, and then patches containing exenatide formulation with hydroxyethyl cellulose (HEC) or hydroxypropyl cellulose (HPC) was affixed to the treated skin, and the level of exenatide in plasma was determined.

To perform the experiment, pigs were subjected to the following treatments:
1) ViaDerm and 5 mg/ml exenatide in 1.5 HEC gel—each pig was treated with the ViaDerm™ instrument for 700 μsec to generate micro-channels at a density of 150 micro-channels/cm². Thereafter, Finn chamber containing 5 mg/ml exenatide in 1.5% HEC gel, buffer acetate 20 mM, pH 4.9-5.5 and trehalose at a ratio 1:5 (w/w peptide:trehalose) was affixed to the treated skin.
2) ViaDerm and 5 mg/ml exenatide in 1.5% HPC gel—each pig was treated with the ViaDerm™ instrument for 700 μsec to generate micro-channels at a density of 150 micro-channels/cm². Thereafter, Finn chamber containing 5 mg/ml exenatide in 1.5% HPC gel, buffer acetate 20 mM, pH 4.9-5.5 and trehalose at a ratio 1:11 (w/w; peptide:trehalose) was affixed to the treated skin.

### Results

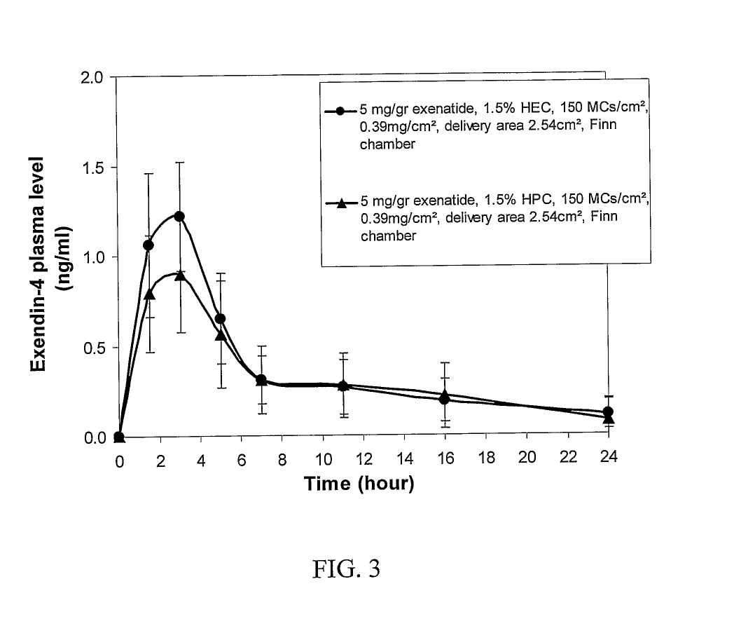

Exenatide plasma levels are shown in FIG. 3. As shown in FIG. 3, the transdermal delivery of exenatide from a formulation containing HEC was higher than from a formulation containing HPC.

### Example 3

### Transdermal Delivery of Exendin-4 Formulated with Hydroxyethyl Cellulose—in Rats

To evaluate whether extended release of exendin-4 can be achieved by transdermal delivery through micro-channels, rats were treated with the ViaDerm apparatus to generate micro-channels, and then a patch containing hydroxyethyl cellulose (HEC) and exendin-4 was affixed to the treated skin, and the level of exendin-4 in plasma was determined. To perform the experiment, rats were subjected to the following treatments:
3) Subcutaneous (SC) injection of 1 μg Exenatide (Byetta, Amylin Pharmaceuticals Inc.).
4) ViaDerm and 1 mg/ml Exendin-4 in 2.5% HEC gel—each rat was treated with the ViaDerm™ instrument for 700 μsec to generate micro-channels at a density of 75 micro-channels/cm². Thereafter, a silicone pouch containing 200 μl of 1 mg/ml exendin-4 (total 200 μg; purchased from CS Bio Menlo Park, Calif., USA or from Shaanxi Zhongbang Pharma-Tech Co., Ltd., China) in 2.5% HEC gel, buffer acetate 10 mM, pH 4.5 and trehalose at a ratio 1:11 (w/w peptide:trehalose) was affixed to the treated skin.
5) ViaDerm and 0.3 mg/ml eξendin-4 in 2.5% HEC gel—each rat was treated with the ViaDerm™ instrument for 700 μsec to generate micro-channels at a density of 75 micro-channels/cm². Thereafter, a silicone pouch containing 200 μl of 0.3 mg/ml exendin-4 (total 60 μg) in 2.5% HEC gel, buffer acetate 10 mM, pH 4.5 and trehalose at a ratio 1:11 (w/w; peptide:trehalose) was affixed to the treated skin.
6) ViaDerm and 0.3 mg/ml exendin-4 in 1% HEC gel—each rat was treated with the ViaDerm instrument for 700 μsec to generate micro-channels at a density of 75 micro-channels/cm². Thereafter, a silicone pouch containing 200 μl of 0.3 mg/ml exendin-4 (total 60 μg) in 1% HEC gel, buffer acetate 10 mM, pH 4.5, and Trehalose at a ratio 1:11 (w/w; peptide:trehalose) was affixed to the treated skin.

Evaluation of the serum levels of exendin-4 was performed by exendin-4 Elisa kit following the instructions provided by the manufacturer (Phonix).

Transepithelial water loss (TEWL) was determined before micro-channel generation and afterwards to confirm that micro-channels were generated.

### Results

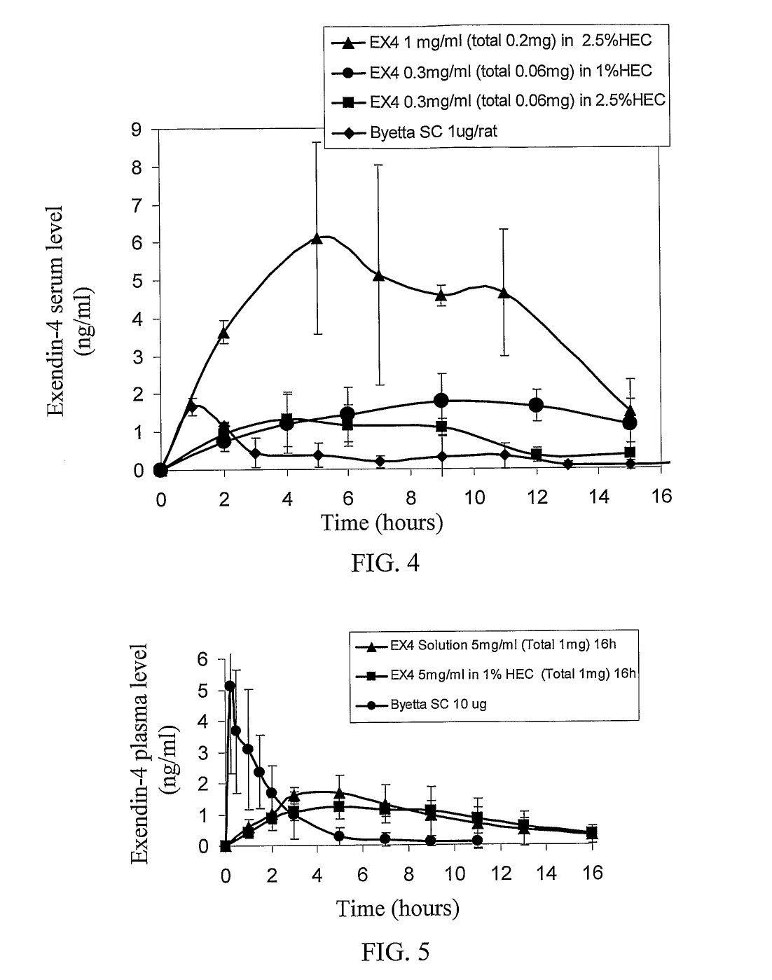

Exendin-4 serum levels are shown in FIG. 4. As shown in FIG. 4, a prolonged release profile for at list 11 hours was observed following transdermal delivery of exendin-4 loaded in gels. In contrast, the SC injection exhibited a peak profile with drug exposure duration of about 3 hours. Increasing the drug concentration from 0.3 to 1 mg/ml resulted in a significant increase of the delivery (FIG. 4). In addition, a more viscous gel (2.5% vs. 1% HEC concentration) resulted in a shorter exendin-4 delivery (12 hours delivery in comparison to at least 16 hours, respectively; FIG. 4). Serum levels of exendin-4 were similar for both 1% and 2.5% HEC up to 6 hours. However, after 6 hours, exendin-4 serum levels were lower in the 2.5% HEC gel group (FIG. 4).

Pharmacokinetics parameters of exendin-4 and its bioavailability are summarized in Table 1.

TABLE 1
Pharmacokinetic parameters of exendin-4 in rats.

| | | AUC | | | | | Amount | |
|---|---|---|---|---|---|---|---|---|
| | | (ng-hr/ml) | | | % | | delivered | |
| | | 16 hr | | | bioavailability | | (μg) 16 hr | |
| Groups study | Rat # | | AVG | SD | AVG | SD | AVG | SD |
| Group #1- | 1 | 6 | 5.67 | 2.52 | 100 | | | |
| SC 1 μg | 2 | 3 | | | | | | |
| | 3 | 8 | | | | | | |
| Group #2- | 4 | 70 | 60.67 | 8.62 | 5.35 | 0.76 | 10.71 | 1.52 |
| Exendin-4 | 5 | 53 | | | | | | |
| 1 mg/ml in | 6 | 59 | | | | | | |
| 2.5% HEC | | | | | | | | |
| (Total 200 μg) | | | | | | | | |
| Group #3- | 7 | 18 | 18.33 | 6.51 | 5.39 | 1.91 | 3.24 | 1.15 |
| Exendin-4 | 8 | 25 | | | | | | |
| 0.3 mg in | 9 | 12 | | | | | | |
| 1% HEC | | | | | | | | |
| (Total 60 μg) | | | | | | | | |
| Group #4- | 10 | 10 | 12.00 | 3.46 | 3.53 | 1.02 | 2.12 | 0.61 |
| Exendin-4 | 11 | 10 | | | | | | |
| 0.3 mg/ml in | 12 | 16 | | | | | | |
| 2.5% HEC | | | | | | | | |
| (Total 60 μg) | | | | | | | | |

Bioavailability was calculated relatively to the averaged AUC obtained from exendin-4 delivered by SC injection. Increasing exendin-4 concentration by ˜3 fold in group #2 vs. group #4 resulted in a ˜5 fold increase of the AUC (AUC of 60 ng-hr/ml during 16 hours vs. 12 ng-hr/ml during 16 hours for 1 mg/ml vs. 0.3 mg/ml exendin-4, 2.5% HEC, respectively). Decreasing the gel viscosity from 2.5% to 1% resulted in a ˜1.5 fold increase of the AUC. The relative bioavailability ranged from 3.5% to 5.4%.

### Example 4

### Transdermal Delivery of Exendin-4 Formulated in Gel or Solution—in Pigs

This study evaluated the transdermal delivery of exendin-4 from patches, in which exendin-4 was formulated with hydroxyethyl cellulose gel or was present in a solution. Micro-channels were generated in pig skin by the ViaDerm™ apparatus and exendin-4 diffused from the patches into the pig circulation.

Pigs were subjected to the following treatments:
1) Subcutaneous (SC) injection of 10 μg Exenatide (Byetta, Amylin Pharmaceuticals Inc.).
2) ViaDerm and 5 mg/ml exendin-4 in 1% HEC gel—Pigs were treated with the ViaDerm™ apparatus to generate micro-channels at a density of 150 micro-channels/cm². Then, a silicone pouch containing 200 μl of 5 mg/ml exendin-4 in 1% HEC, 10 mM acetate buffer pH 4.5 and trehalose at a ratio of 1:11 (w/w; peptide:trehalose) was affixed to the treated skin for 16 hours.
3) ViaDerm and 5 mg/ml exendin-4 solution—Pigs were treated with the ViaDerm™ apparatus to generate micro-channels at a density of 150 micro-channels/cm². Then, a silicone pouch containing 200 μl of 5 mg/ml exendin-4 in 10 mM acetate buffer pH 4.5 and trehalose at a ratio of 1:11 (w/w; peptide:trehalose) was affixed to the treated skin for 16 hours.

Determination of exendin-4 in pig plasma was performed as described in Example 1. Transepithelial water loss (TEWL) was determined before micro-channel generation and afterwards to confirm that micro-channels were generated.

### Results

FIG. 5 shows the delivery of exendin-4 from a solution and from a 1% HEC gel. As shown in FIG. 5, the delivery from the solution and HEC gel resulted in a prolonged drug delivery in comparison to a typical peak profile obtained by SC injection. The plasma exendin-4 concentrations obtained following application of the gel formulation were maintained at ˜1 ng/ml for 9 hours (from 2-11 hours post application) with relatively small standard deviation in comparison to the SC treatment.

The pharmacokinetic parameters of this experiment are summarized in Table 2.

TABLE 2
Pharmacokinetic parameters of exendin-4 in pigs.

| | | Cmax | | | AUC | | | % | | | Amount | | |
|---|---|---|---|---|---|---|---|---|---|---|---|---|---|
| | | (ng/ml) | | | (ng-hr/ml) | | | bioavailability | | | delivered (μg) | | |
| Group | Pig # | | Avg | stdev | | Avg | stdev | | Avg | Stdev | | Avg | Stdev |
| Group #1- | 1 | 8.00 | 5.13 | 2.81 | 11 | 9.25 | 6.02 | 100.00 | | | 10.00 | | |
| SC 10ug | 2 | 7.07 | | | 17 | | | | | | | | |
| | 3 | 3.05 | | | 5 | | | | | | | | |
| | 4 | 2.41 | | | 4 | | | | | | | | |
| Group #2- | 1 | 0.97 | 1.40 | 0.34 | 10 | 13.75 | 2.63 | 0.91 | 2.09 | 1.36 | 9.09 | 20.88 | 13.58 |
| 5 mg/ml in | 2 | 1.37 | | | 16 | | | 0.94 | | | 9.41 | | |
| 1% HEC - | 3 | 1.46 | | | 15 | | | 3.00 | | | 30.00 | | |
| Total 1 mg | 4 | 1.81 | | | 14 | | | 3.50 | | | 35.00 | | |
| Group #3- | 1 | 1.94 | 1.99 | 0.36 | 19 | 15.00 | 5.60 | 1.73 | 2.27 | 1.87 | 17.27 | 22.73 | 18.67 |
| Solution | 2 | 2.51 | | | 13 | | | 0.76 | | | 7.65 | | |
| 5 mg/ml | 3 | 1.70 | | | 8 | | | 1.60 | | | 16.00 | | |
| 16 h | 4 | 1.81 | | | 20 | | | 5.00 | | | 50.00 | | |
| Total 1 mg | | | | | | | | | | | | | |

As shown in Table 2, no differences were found between the transdermal delivery of exendin-4 from solution and from HEC gel (Cmax of 2.0 vs. 1.4 ng/ml; AUC of 15.0 vs. 13.8 ng-hr/ml, respectively). The bioavailability of exendin-4 in the ViaDerm treated groups as calculated in relation to the SC injected group was ˜2% and the amount of exendin-4 delivered was ˜20 μg.

### Example 5

Transdermal Delivery of Exendin-4 Formulated with 1% Hydroxyethyl Cellulose—in Pigs

The present experiment was aimed at evaluating the effect of different micro-channel densities as well as exendin amounts applied.

The treatments were as follows:
1) Subcutaneous (SC) injection of 5 μg Exenatide (Byetta, Amylin Pharmaceuticals, Inc.);
2) ViaDerm and 5 mg/ml exendin-4 in 1% HEC gel, 150 micro-channels/cm²—Pigs were treated with the ViaDerm™ apparatus to generate micro-channels at a density of 150 micro-channels/cm². Then, a silicone pouch containing 200 μl of 5 mg/ml exendin-4 in 1% HEC, 10 mM acetate buffer pH 4.5 and trehalose at a ratio of 1:11 (w/w; peptide:trehalose) was affixed to the treated skin for 16 hours. Total dose: 694 μg/cm;
3) ViaDerm and 5 mg/ml exendin-4 in 1 HEC gel, 300 micro-channels/cm²—Pigs were treated with the ViaDerm™ apparatus to generate micro-channels at a density of 300 micro-channels/cm². Then, a silicone pouch containing 200 μl of 5 mg/ml exendin-4 in 1% HEC, 10 mM acetate buffer pH 4.5 and trehalose at a ratio of 1:11 (w/w; peptide:trehalose) was affixed to the treated skin for 16 hours. Total dose: 694 μg/cm;
4) ViaDerm and 2.5 mg/ml exendin-4 in 1 HEC gel, 300 micro-channels/cm²—Pigs were treated with the ViaDerm™ apparatus to generate micro-channels at a density of 300 micro-channels/cm². Then, a silicone pouch containing 200 μl of 2.5 mg/ml exendin-4 in 1% HEC, 10 mM acetate buffer pH 4.5 and trehalose at a ratio of 1:11 (w/w; peptide:trehalose) was affixed to the treated skin for 16 hours. Total dose: 347 μg/cm;
5) Alzet® [model 2001D (8.0 μl per hour, 1 day), purchased from DURECT Corporation, Cupertino, Calif., USA. mini osmotic pump at rate of 5 μg/h for an effective delivery period of 10 hours.

Transepithelial water loss (TEWL) was determined before micro-channel generation and afterwards to confirm that micro-channels were generated. Two control groups were tested in this experiment: SC group which represents short duration of drug exposure, and Alzet® mini osmotic pump which represents continuous drug delivery (10 hours).

### Results

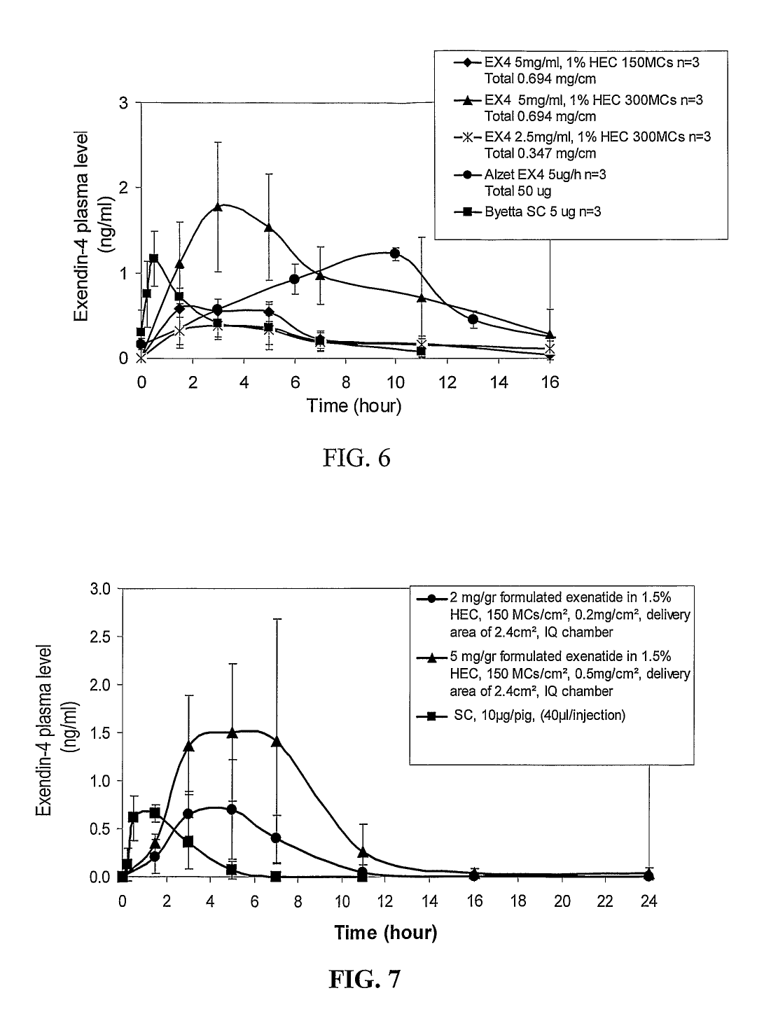

FIG. 6 shows the results of the experiment performed on pigs. As shown in FIG. 6 and in Table 3, doubling the micro-channel density resulted in 3 fold increase of bioavailability, i.e., the AUC was 14.67±6.51 for 300 MCs vs. 4.67±0.58 ng-hr/ml for 150 MCs study group. Increasing exendin-4 amount by 2 fold resulted in 2 fold increase of bioavailability (Table 3). It should be emphasized that bioavailability was calculated in comparison to SC delivery and to osmotic mini pump delivery. The results indicated that the bioavailability for each ViaDerm treatment group was 3 fold higher when compared to Alzet mini osmotic pump than to SC injection. Without wishing to be bound to any mechanism of action, this may indicate that exendin administered by mini osmotic pump undergoes metabolism in the skin, a process that presumably does not occur for SC injected peptide. Therefore, the control of mini osmotic pump was chosen as a representative control for transdermal delivery of this peptide.

TABLE 3
Pharmacokinetic parameters of exendin-4.

| | | | | | | % | | % | |
|---|---|---|---|---|---|---|---|---|---|
| | | | | AUC | | bioavailability | | bioavailability | |
| | | | | (ng- | | 16 hr* | | 16 hr | |
| | Total | Cmax | | hr/ml) | | compared to | | compared to | |
| | dose | (ng/ml) | | 16 hr | | alzet | | SC | |
| Group | (ug/cm) | Avg | stdev | Avg | stdev | Avg | stdev | Avg | stdev |
| Group #1- | 5 | 1.22 | 0.30 | 4.33 | 1.15 | 100.00 | | 100.00 | |
| SC 5ug | | | | | | | | | |
| Group #2- | 694 | 0.77 | 0.24 | 4.67 | 0.58 | 2.83 | 0.55 | 0.83 | 0.33 |
| 5 mg/ml, | | | | | | | | | |
| 1% HEC | | | | | | | | | |
| 150MCs | | | | | | | | | |
| Group #3- | 694 | 1.78 | 0.76 | 14.67 | 6.51 | 8.95 | 3.68 | 2.75 | 1.38 |
| 5 mg/ml, | | | | | | | | | |
| 1% HEC | | | | | | | | | |
| 300MCs | | | | | | | | | |
| Group #4- | 347 | 0.40 | 0.17 | 3.33 | 1.15 | 4.08 | 1.63 | 1.02 | 0.39 |
| 2.5 mg/ml, | | | | | | | | | |
| 1% HEC | | | | | | | | | |
| 300MCs | | | | | | | | | |
| Group #5- | 50 | 1.22 | 0.07 | 12.00 | 1.00 | | | | |
| Alzet | | | | | | | | | |
| 5 ug/h | | | | | | | | | |

### Example 6

### Transdermal Delivery of Exenatide Formulated with 1.5% Hydroxyethyl Cellulose—in Pigs

Transdermal delivery of exenatide formulated with 1.5% (w/w) HEC was determined after generation of micro-channels in pigs with the ViaDerm™ apparatus. The level of exenatide in plasma was determined.

To perform the experiment, pigs were subjected to the following treatments:
1. ViaDerm and 2 mg/ml exenatide in 1.5% HEC gel—each pig was treated with the ViaDerm™ instrument for 700 μsec to generate micro-channels at a density of 150 micro-channels/cm². Thereafter, IQ chamber containing 2 mg/ml exenatide in 1.5% HEC gel, buffer acetate 20 mM, pH 4.9-5.5, trehalose at a ratio 1:5 (w/w peptide:trehalose), and 2.2 mg M-Cresol/gr gel was affixed to the treated skin.
2. ViaDerm and 5 mg/ml exenatide in 1.5% HPC gel—each pig was treated with the ViaDerm™ instrument for 700 μsec to generate micro-channels at a density of 150 micro-channels/cm². Thereafter, IQ chamber containing 5 mg/ml exenatide in 1.5% HPC gel, buffer acetate 20 mM, pH 4.9-5.5, trehalose at a ratio 1:5 (w/w; peptide:trehalose), and 2.2 mg M-Cresol/gr gel was affixed to the treated skin.
3. Subcutaneous (SC) injection of 5 □g Exenatide (Byetta, Amylin Pharmaceuticals, Inc.)

### Results

Exenatide plasma levels are shown in FIG. 7. As shown in FIG. 7, the transdermal delivery of exenatide from HEC gel increased with the increase of exenatide concentration in the gel.

### Example 7

### Effect of Transdermal Delivery of Exenatide on Food Intake in Pigs

The effect of transdermal delivery or subcutaneous (SC) injection of exenatide to pigs on food intake was evaluated. The experiment was conducted as detailed in Example 6 herein above, except that the amount of exenatide injected SC was 20 μg, and food intake was measured. The results indicated that transdermal delivery of exenatide resulted in a decrease in food intake compared to a control group which was not treated with exenatide. Moreover, the decrease in food intake observed in the ViaDerm treated group lasted for a longer period of time than that observed in pigs injected subcutaneously with exenatide.

It will be appreciated by persons skilled in the art that the present invention is not limited by what has been particularly shown and described herein above. Rather the scope of the invention is defined by the claims that follow.

1.-37. (canceled)

38. A transdermal patch formulation comprising an incretin or incretin mimetic peptide, a stabilizer, a buffer, a water soluble thickening agent, and a pharmaceutically acceptable carrier, wherein the formulation is in the form of a viscous liquid.

39. The transdermal patch formulation according to claim 38, wherein the incretin or incretin mimetic peptide is selected from the group consisting of an exendin, GLP-1, PYY, PP, amylin, pramlintide, and an analog, fragment, or conjugate thereof.

40. The transdermal patch formulation according to claim 39, wherein the exendin is selected from exendin-4, exendin-3, and an analog or fragment thereof of the amino acid sequence as set forth in any one of SEQ ID NO: 1 to 8.

41. The transdermal patch formulation according to claim 39, wherein the exendin is exendin-4 of the amino acid sequence as set forth in SEQ ID NO: 1.

42. The transdermal patch formulation according to claim 38, wherein the incretin or incretin mimetic peptide is of the amino acid sequence as set forth in any one of SEQ ID NOs: 9 to 15.

43. The transdermal patch formulation according to claim 38, wherein the stabilizer is a simple or complex carbohydrate.

44. The transdermal patch formulation according to claim 38, wherein the stabilizer is trehalose.

45. The transdermal patch formulation according to claim 38, wherein the buffer is selected from the group consisting of acetate buffer, citrate buffer, glutamate buffer, and phosphate buffer.

46. The transdermal patch formulation according to claim 38, wherein the buffer is acetate buffer.

47. The transdermal patch formulation according to claim 38, wherein the water soluble thickening agent is a water soluble etherified cellulose derivative selected from the group consisting of a hydroxyalkyl cellulose, alkyl cellulose, and alkylhydroxyalkyl cellulose.

48. The transdermal patch formulation according to claim 38, wherein the water soluble thickening agent is hydroxyethyl cellulose.

49. The transdermal patch formulation according to claim 38, wherein the water soluble thickening agent is present in the formulation in an amount ranging from about 0.5% (w/w) to about 3.5% (w/w) of the formulation.

50. The transdermal patch formulation according to claim 38, comprising exendin-4 as set forth in SEQ ID NO:1, trehalose, acetate buffer, wherein the acetate buffer maintains the pH of the formulation in the range of about 4.9 to about 5.5, hydroxyethylcellulose in an amount ranging from about 1% (w/w) to about 3% (w/w) of the formulation, and water.

51. A patch adapted for transdermal delivery of an incretin or incretin mimetic peptide, the patch comprises a drug reservoir compartment comprising the transdermal patch formulation according to claim 38.

52. A system for facilitating transdermal delivery of an incretin or incretin mimetic peptide through skin of a subject comprising: (i) an apparatus capable of generating a plurality of micro-channels in an area on the skin of the subject; and (ii) a patch comprising a drug reservoir compartment comprising a transdermal patch formulation according to claim 38.

53. A method for extended transdermal delivery of an incretin or incretin mimetic peptide comprising:
generating a plurality of micro-channels in an area on the skin of a subject;
affixing a patch according to claim 51 to the area of skin in which the plurality of micro-channels is present; and
achieving a therapeutic plasma concentration of the incretin or incretin mimetic peptide for an extended period of time.

54. The method of claim 53, wherein the patch is affixed to the skin of a subject having diabetes mellitus for reducing blood glucose level.

55. The method of claim 53, wherein the patch is affixed to the skin of a subject for lowering plasma glucagon level therein.

56. The method of claim 53, wherein the patch is affixed to the skin of a subject for assisting the subject in reducing food intake.

57. The method of claim 53, wherein the patch is affixed to the skin of a subject for reducing gastric motility therein.


SEQUENCE LISTING (15 sequences)

SEQ ID NO: 1 - protein, 39 aa, Artificial Sequence
  Synthetic peptide
HGEGTFTSDLSKQMEEEAVRLFIEWLKNGGPSSGAPPPS

SEQ ID NO: 2 - protein, 39 aa, Artificial Sequence
  Synthetic peptide
HSDGTFTSDLSKQMEEEAVRLFIEWLKNGGPSSGAPPPS

SEQ ID NO: 3 - protein, 30 aa, Artificial Sequence
  Synthetic peptide
HGEGTFTSDLSKQMEEEAVRLFIEWLKNGG

SEQ ID NO: 4 - protein, 30 aa, Artificial Sequence; identical to SEQ ID NO: 3
  Synthetic peptide

SEQ ID NO: 5 - protein, 28 aa, Artificial Sequence
  Synthetic peptide
HGEGTFTSDLSKQMEEEAVRLFIEWLKN

SEQ ID NO: 6 - protein, 39 aa, Artificial Sequence
  Synthetic peptide
HGEGTFTSDLSKQLEEEAVRLFIEFLKNGGPSSGAPPPS

SEQ ID NO: 7 - protein, 28 aa, Artificial Sequence
  Synthetic peptide
HGEGTFTSDLSKQLEEEAVRLFIEFLKN

SEQ ID NO: 8 - protein, 28 aa, Artificial Sequence
  Synthetic peptide
HGEGTFTSDLSKQLEEEAVRLAIEFLKN

SEQ ID NO: 9 - protein, 36 aa, Artificial Sequence
  Synthetic peptide
HDEFERHAEGTFTSDVSSYLEGQAAKEFIAWLVKGR

SEQ ID NO: 10 - protein, 42 aa, Homo sapiens
YAEGTFISDYSIAMDKIHQQDFVNWLLAQKGKKNDWKHNITQ

SEQ ID NO: 11 - protein, 34 aa, Artificial Sequence
  Synthetic peptide
IKPEAPGEDASPEELNRYYASLRHYLNLVTRQRY

SEQ ID NO: 12 - protein, 34 aa, Artificial Sequence; identical to SEQ ID NO: 11
  Synthetic peptide

SEQ ID NO: 13 - protein, 36 aa, Artificial Sequence
  Synthetic peptide
APLEPVYPGDNATPEQMAQYAADLRRYINMLTRPRY

SEQ ID NO: 14 - protein, 62 aa, Homo sapiens
HQVEKRKCNTATCATQRLANFLVHSSNNFGAILSSTNVGSNTYGKRNAVEVLKREPLNYLPL

SEQ ID NO: 15 - protein, 37 aa, Artificial Sequence
  Synthetic peptide
KCNTATCATQRLANFLVHSSNNFGPILPPTNVGSNTY